(19) United States
(12) Patent Application Publication
(10) Pub. No.: US 2025/0377154 A1
(43) Pub. Date: Dec. 11, 2025

(54) AERODYNAMIC BLAST FREEZE CONTAINERS

(71) Applicant: ENTEGRIS, INC., Billerica, MA (US)

(72) Inventors: Robert M. Shydo, Jr., Hampstead, NH (US); Troy Galanis, North Billerica, MA (US); Jessie M. Geannaris, Londonderry, NH (US)

(21) Appl. No.: 19/231,294

(22) Filed: Jun. 6, 2025

Related U.S. Application Data
(60) Provisional application No. 63/658,832, filed on Jun. 11, 2024.

(51) Int. Cl. F25D 25/00; A01N 1/162; B65D 21/02

(52) U.S. Cl. CPC F25D 25/005; B65D 21/0215; A01N 1/162

(57) ABSTRACT

Aerodynamic members and/or spacers are provided to improve airflow around shells used in processes such as blast freezing. The aerodynamic members can include air control surfaces configured to control airflow so as to reduce a boundary layer of flow over a blast freeze container. The spacers can provide spacing between adjacent blast freeze containers. The spacers and/or aerodynamic members can be incorporated into the blast freeze containers as attachments or integral structures of the containers. Aerodynamic members and spacers can also be combined into articles for placement between blast freeze containers.

### FIELD

This disclosure is directed to blast freeze containers, particularly including spacers and/or aerodynamic features for improving flow over the containers.

### BACKGROUND

Bags, such as bioprocess bags can be blast-frozen in blast freeze containers that are placed in a refrigerated space where cold air is directed over the containers. Such bags can sometimes be thawed within such containers, for example within thawing apparatuses, such as warm air convection apparatuses. Blast freeze containers typically provide flat faces perpendicular to the airflow within the blast freezer or thawing apparatus. The airflow around such shapes tends to form large boundary layers.

### SUMMARY

This disclosure is directed to blast freeze containers, particularly including spacers and/or aerodynamic features for improving flow over the containers.

By providing aerodynamic features and/or controlling the spacing of blast freeze containers, the thickness of boundary layers of a cooling or thawing airflow over the blast freeze containers can be reduced, thereby improving flow and heat transfer to the blast freeze containers. The speed and consistency of freezing bioprocess bags in the containers can thereby be improved by providing one or both of the aerodynamic features and spacers between adjacent blast freeze containers. Spacers can be provided to ensure a gap of a predetermined size between adjacent container shells, which the cooling or thawing airflow passes through when freezing or thawing the contents of the container. Aerodynamic features can influence flow so as to reduce the boundary layer thickness.

In an embodiment, an apparatus includes a shell including a front face configured to face a source of an airflow in a blast freezer and a rear face, opposite the front face, an upper surface, and a lower surface. The apparatus further includes an aerodynamic member positioned at one of the front face or the rear face, the aerodynamic member configured to present an airflow control surface on said one of the front face or the rear face.

In an embodiment, the apparatus further includes one or more spacers positioned at an upper surface and/or a lower surface of the shell. In an embodiment, the one or more spacers are configured to separate the shell from an adjacent shell by a predetermined distance. In an embodiment, the one or more spacers are provided on the lower surface of the shell.

In an embodiment, the aerodynamic member is formed integrally with the shell.

In an embodiment, the aerodynamic member is attached to the shell.

In an embodiment, the aerodynamic member is formed of a polymer.

In an embodiment, the aerodynamic member includes a hollow cavity.

In an embodiment, the aerodynamic member is positioned at the front face.

In an embodiment, an apparatus includes a shell. The shell includes a front face configured to face a source of an airflow in a blast freezer and a rear face, opposite the front face; an upper surface, and a lower surface. The apparatus further includes one or more spacers provided on the shell, the one or more spacers configured to separate the shell from an adjacent shell.

In an embodiment, the one or more spacers are configured to separate the shell from said adjacent shell by a distance in a range from 0.5 inches to 4 inches.

In an embodiment, the one or more spacers are provided on the lower surface of the shell.

In an embodiment, the apparatus further includes an aerodynamic member positioned at one of the front face or the rear face, the aerodynamic member configured to present an air control surface on said one of the front face or the rear face. In an embodiment, the aerodynamic member is formed integrally with the shell. In an embodiment, the aerodynamic member is attached to the shell. In an embodiment, the aerodynamic member is formed of a polymer. In an embodiment, the aerodynamic member includes a hollow cavity. In an embodiment, the aerodynamic member is positioned at the front face.

In an embodiment, an apparatus includes an aerodynamic member including an air control surface and one or more spacers configured to contact a shell, wherein the apparatus is configured to accommodate the shell such that the aerodynamic member is provided at a front face or a rear face of the shell.

In an embodiment, the one or more spacers are further configured to contact a second shell, such that the shell and the second shell are spaced apart by a predetermined distance, and the shell and the second shell are oriented in a same direction.

### BRIEF DESCRIPTION OF THE DRAWINGS

FIG. 1 shows an exploded view of a container shell according to an embodiment.

FIG. 2 shows a plurality of container shells according to an embodiment within a temperature control apparatus.

FIG. 3 shows an aerodynamic member for use with a container shell according to an embodiment.

FIG. 4 shows a spacer for use with a container shell according to an embodiment.

FIG. 5 shows a spacer and aerodynamic member assembly according to an embodiment.

FIG. 6 shows a method of blast freezing a bioprocess material.

FIG. 7 shows a box plot of freezing times for bags in freezing containers according to an embodiment.

### DETAILED DESCRIPTION

This disclosure is directed to blast freeze containers, particularly including spacers and/or aerodynamic features for improving flow over the containers.

As used herein, an “aerodynamic member” is a member providing an air control surface in a position where the air control surface will be contacted by an airflow.

As used herein, an “air control surface” is a surface configured to affect an airflow to achieve desired characteristics for said airflow, for example by deflecting, directing, diverting, dividing, combining, or otherwise affecting one or more airflows passing over said air control surface. The characteristics of the airflow can include speed, direction, turbulence or laminarity of flow, boundary layer thickness, and the like. Air control surfaces can include one or more flat surfaces, one or more curved surfaces, combinations thereof, and the like. Where different air control surfaces meet, the intersection of the surfaces can be a sharp corner, rounded, or any other suitable intersection of surfaces.

As used herein, being oriented in “a same direction” is when the major axes of each item so oriented in the same direction are substantially parallel to one another. As used herein, “substantially parallel” is where deviations from being parallel result from variations within tolerances, the presence of manufacturing defects, or the like.

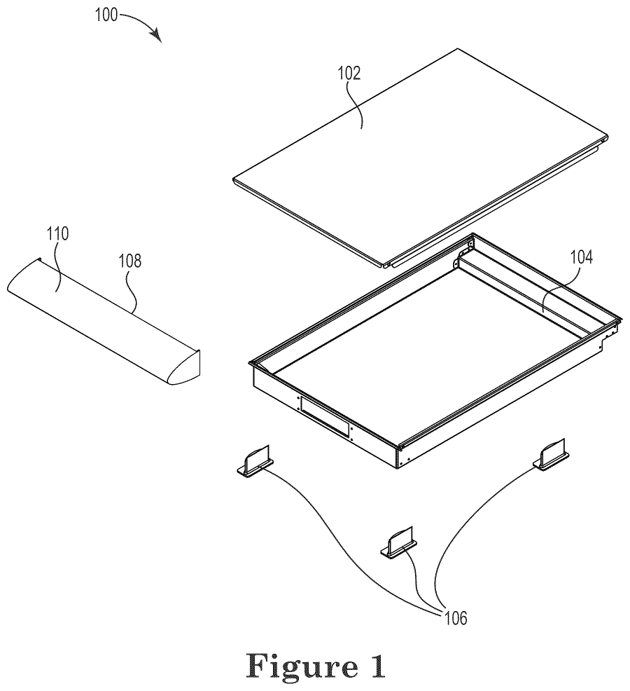

FIG. 1 shows an exploded view of a container shell according to an embodiment. Container shell 100 includes first container segment 102, second container segment 104, spacers 106 and an aerodynamic member 108.

Container shell 100 is a shell configured to accommodate a bag such as a bioprocess bag, for example during freezing and/or thawing processes. The container shell 100 can be, for example, a blast freezing shell for use in a blast freezer. In an embodiment, the container shell 100 is formed of metal. Container shell 100 can include a first container segment 102 and a second container segment 104 that are configured to be combined to define an internal space capable of accommodating the bag. Container shell 100 can be configured to fit within a blast freezer, thawing apparatus, refrigerator, or the like. Container shell 100 can have a first end configured to face an airflow provided by the blast freezer, thawing apparatus, refrigerator, or the like, a second end opposite the first end, and sides extending from the first end to the second end. The container shell 100 can have the shape of a rectangular prism, with the sides, ends, and upper and lower surfaces respectively meeting at right angles. In an embodiment, the surfaces of first container segment and second container segments 102, 104 at the first and/or second ends can be generally flat surfaces. The container shell 100 can be configured such that the first and second ends defined by the first and second container segments 102, 104 have planes at or near perpendicular to an airflow when positioned within a blast freezer, thawing apparatus, refrigerator, or the like.

Spacers 106 are one or more members provided on at least one of the first container segment 102 and the second container segment 104 so as to space the first container segment 102 and/or second container segment 104 apart from container segments of an adjacent container shell. Spacers 106 can be one or more blocks or other structures providing two opposing surfaces having a predetermined thickness between said opposing surfaces, each surface shaped and/or positioned to support or be supported by an adjacent container shell to the container shell 100. The predetermined thickness between the opposing surfaces of spacers 106 can be selected to as to achieve a desired spacing between the container shell 100 and an adjacent container shell. The desired spacing can be selected based on aerodynamic properties of flow between the container shell 100 and the adjacent container shell, for example pressure differential, flow velocity, turbulence, boundary layer thickness, or any other suitable property of flow between the container shells that can be affected by the spacing of said container shells. The spacers 106 can be attached to the first container segment 102 or the second container segment 104. The spacers 106 can alternatively be placed, by automation or manually, between an adjacent container shell and the container shell 100.

Aerodynamic member 108 is provided at an end of the first container segment 102 and second container segment 104. In an embodiment, aerodynamic member 108 is provided at an end of the first and second container segments 102, 104 configured to face towards a source of an airflow over the container shell 100, such as a blast freezer, thawing apparatus, refrigerator, or the like. In an embodiment, aerodynamic member 108 is provided at an end of the first and second container segments 102, 104 configured to be opposite the source of an airflow over the container shell 100. In an embodiment, aerodynamic member 108 is attached to the end of the first and second container segments 102, 104. In an embodiment, aerodynamic member 108 is mechanically fit to the end of the first and second container segments 102, 104. Aerodynamic member 108 includes an air control surface 110 on an exterior of the aerodynamic member 108. Air control surface 110 can be any suitable shape for affecting an airflow over the container shell 100 to achieve improved or desired properties of said airflow. Air control surface 110 can be configured to deflect, direct, divert, divide, combine, and/or otherwise affect the airflow over the container shell 100, for example when an airflow is provided over the container shell 100 by a blast freezer, thawing apparatus, refrigerator, or the like. Air control surface 110 can be configured to, for example, reduce a boundary layer of airflow over the container shell 100 compared to the boundary layer when the same airflow is provided over first and second container segments 102, 104 in the absence of the aerodynamic member 108. A non-limiting example of air control surface 110 is the curved outer surface of aerodynamic member 108, as shown in FIG. 1.

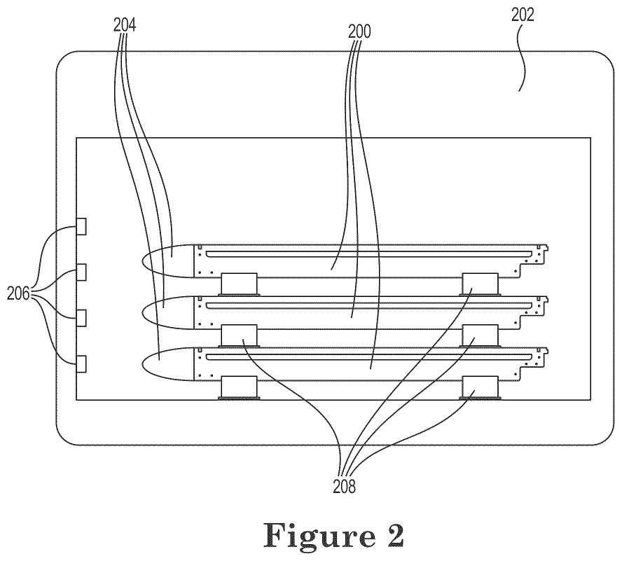

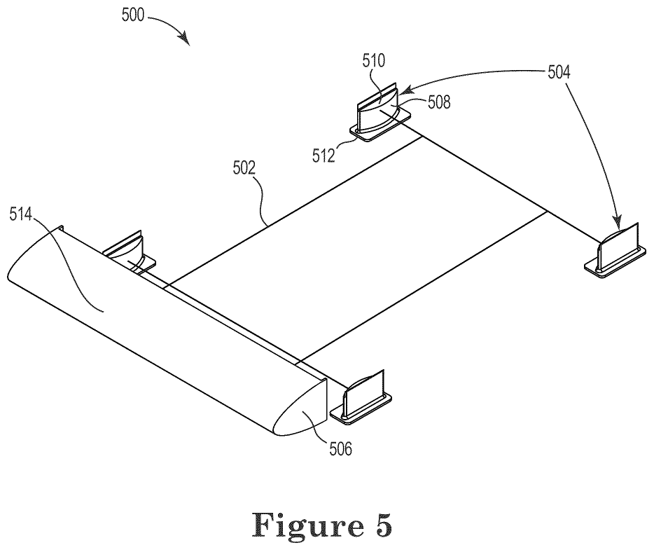

FIG. 2 shows a plurality of container shells according to an embodiment within a temperature control apparatus. The container shells 200 are each a container shell containing a bioprocess bag (not shown) to be frozen in the temperature control apparatus 202. Each of the container shells can have an aerodynamic member 204 positioned towards cold air outlets 206 of the temperature control apparatus 202. Aerodynamic member(s) 204 provide an air control surface configured to influence flow from the cold air outlets 206. The aerodynamic member 204 can be configured to reduce a boundary layer thickness of the flow of cold air from outlets 206 over the container shells 200. Spacers 208 are disposed between the container shells 200. In an embodiment, when the container shells are supported by spacers 208, the major axes of adjacent container shells 200 can be substantially parallel. As can be seen in FIG. 2, spacers 208 can provide a consistent spacing between adjacent container shells 200, allowing airflow between said adjacent container shells 200. The spacing of the container shells 200 by spacers 208 can be a distance selected to achieve desired flow properties for the flow of cold air from outlets 206, for example further adjusting boundary layer thickness, achieving desired pressure and/or flow rate characteristics, and the like. In an embodiment, the aerodynamic member 204 and/or spacers 208 are attached to respective container shells 200. A non-limiting example of the container shell 200 including aerodynamic member 204 and spacers 208 is the container shell 100 as described above and shown in FIG. 1. In an embodiment, the spacers 208 and/or the aerodynamic member 204 can be separate elements attached to or positioned in proximity to the respective container shells 200. In an embodiment, spacers 208 and aerodynamic member 204 can be provided in a spacer and aerodynamic member assembly such as, as a non-limiting example, the assembly 500 as described below and shown in FIG. 5.

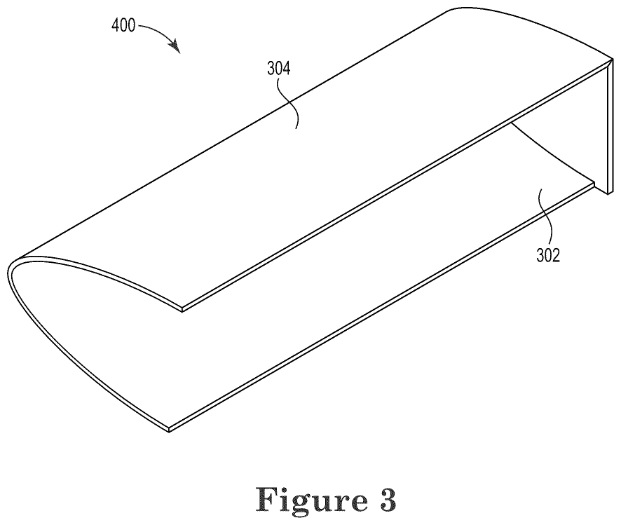

FIG. 3 shows an aerodynamic member for use with a container shell according to an embodiment. Aerodynamic member 300 includes body 302 and air control surface 304.

Aerodynamic member 300 is configured to be positioned at an end of a container shell so as to affect the airflow over said container shell. Aerodynamic member 300 can be made of any suitable material, such as a polymer material. In an embodiment, aerodynamic member 300 can be made of a material selected for the ability to be used at temperatures used in freezing of the container shell without suffering damage or undue wear due to the temperatures and/or repeated freeze-thaw cycles. In an embodiment, aerodynamic member 300 can be composed of multiple segments. In an embodiment, each of the segments can be attached to the container shell separately to cover an end of the container shell. In an embodiment, aerodynamic member 300 can be applied to an end of the container shell facing an airflow provided in a cooling or heating unit such as a blast-freezer. In an embodiment, aerodynamic member 300 can be applied to an end of the container shell opposite where an airflow is provided in the cooling or heating unit. Aerodynamic member 300 can be produced through any suitable manufacturing method for the materials used therein, such as three-dimensional printing, injection molding, stamping of sheet metal, or the like.

Body 302 is configured to present air control surface 304. The body 302 can include an end configured to fit over, engage with, and/or cover an end of a corresponding container shell. The end of body 302 can be open. In an embodiment, body 302 includes a hollow portion, for example to reduce weight, reduce materials usage in manufacturing, or the like. In an embodiment, the open end and the hollow portion of body 302 can be continuous with one another, for example as shown in FIG. 3.

Air control surface 304 is provided on an exterior surface of aerodynamic member 300. The air control surface 304 can be any suitable shape for affecting an airflow over a container shell used with aerodynamic member 300, so as to achieve improved or desired properties of said airflow. Air control surface 304 can be configured to deflect, direct, divert, divide, combine, and/or otherwise affect the airflow over the container shell, for example when an airflow is provided over the container shell by a blast freezer, thawing apparatus, refrigerator, or the like. Air control surface 304 can be configured to, for example, reduce a boundary layer of airflow over the container shell compared to the boundary layer when the same airflow is provided over the same container shell in the absence of the aerodynamic member 300. A non-limiting example of air control surface 304 is the curved outer surface of aerodynamic member 300 as shown in FIG. 3.

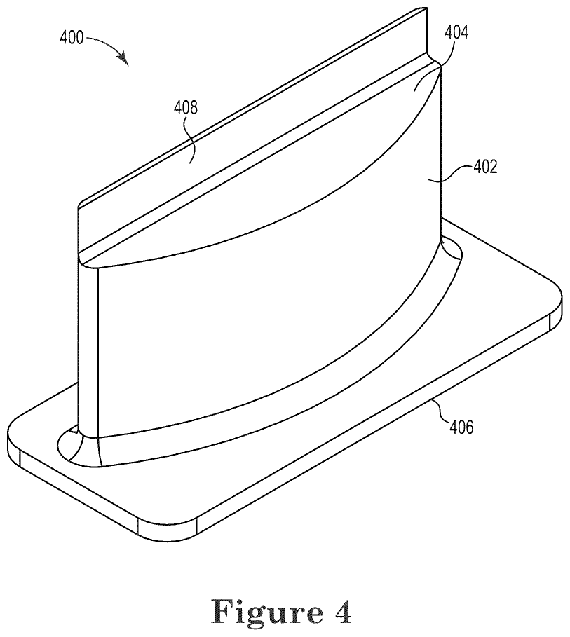

FIG. 4 shows a spacer for use with a container shell according to an embodiment. Spacer 400 includes spacer body 402 having upper contact surface 404 and lower contact surface 406. Spacer body 402 can be formed of any suitable material, such as polymer, metal, or the like. Spacer body 402 can be formed through any suitable manufacturing method, for example machining, injection molding, three-dimensional printing, or the like. Spacer body 402 is configured to provide upper contact surface 404 and lower contact surface 406. Upper contact surface 404 is configured to at least partially support a first one of two adjacent container shells in a stack of container shells, for example when the container shells are stacked within a blast freezer. The upper contact surface 404 is configured to contact a bottom surface of the container shell higher up in the stack of container shells. Lower contact surface 406 is provided opposite the upper contact surface 404. Lower contact surface 406 is configured to contact a second one of the two adjacent container shells, different from the container shell contacting upper contact surface 404. Lower contact surface 406 is configured to contact a top surface of the container shell lower in the stack of container shells. In an embodiment, the respective planes of upper contact surface 404 and lower contact surface 406 are substantially parallel. Spacer body 402 can be configured such that the upper contact surface 404 and the lower contact surface 406 are separated by a predetermined distance, with the predetermined distance being based on desired flow characteristics for a flow between the adjacent container shells, such as a flow of cold air provided in a blast freezer. Examples of flow characteristics on which the predetermined distance can be based include, as non-limiting examples, pressure drop, flow rate, boundary layer thickness, or the like. In an embodiment, the spacer body 402 can include one or more alignment portions 408 outside of the upper and lower contact surfaces 404, 406. The alignment portions 408 can be configured to align the adjacent container shells relative to one another, for example to align the major axes of the adjacent container shells such that said major axes are substantially parallel to one another.

In an embodiment, a plurality of spacers 400 can be provided on or attached to a container shell, for example through adhesive, one or more engagement features provided on the upper contact surfaces 404 or lower contact surfaces 406 of the respective spacers 400. In an embodiment, the spacers 400 can be positioned between adjacent container shells during the stacking thereof, either through automation or manual positioning. In an embodiment, the spacers 400 can be used in combination with one or more aerodynamic members such as aerodynamic members 300 described above and shown in FIG. 3. In an embodiment, the predetermined distance can be based on the flow characteristics when spacers 400 and aerodynamic members 300 are used together.

FIG. 5 shows a spacer and aerodynamic member assembly according to an embodiment. Assembly 500 includes frame 502, spacer blocks 504, and at least one aerodynamic member 506.

Assembly 500 is configured to be positioned between container shells in a freezing apparatus such as a blast freezer, a thawing apparatus, a refrigerator, or the like. Assembly 500 can be placed between container shells manually or by way of automation when the container shells are being stacked in the blast freezer, thawing apparatus, refrigerator, or the like. Assembly 500 can be made of any suitable material such as one or more polymers, metal, or the like. Assembly 500 can be composed of multiple pieces or can be formed as a single unitary piece. Where assembly 500 is composed of multiple pieces, the pieces can be attached through any suitable connections such as mechanical connections, fasteners, adhesives, combinations thereof, and the like. Assembly 500 can be formed through any suitable method for the components and materials used, such as machining, injection molding, or the like.

Frame 502 is configured to position and connect the spacer blocks 504 and the at least one aerodynamic member 506 in suitable positions. The frame 502 can be configured to present the spacer blocks 504 such that the respective upper surfaces of each of the spacer blocks 504 are in plane with one another. The frame 502 can be configured to present the spacer blocks 504 such that the respective lower surfaces of each of the spacer blocks 504 are in plane with one another. The frame 502 can be configured to present the aerodynamic member 506 at or near an end of the container shell to be used with assembly 500. The frame 502 can be configured such that the aerodynamic member 506 does not interfere with the container shell when the container shell is in contact with the spacer blocks 504. In an embodiment, frame 502 can be configured to provide aerodynamic members 506 at each of opposing ends of the container shell. The space between such opposing aerodynamic members can be defined by frame 502 such that the container shell can be accommodated between the aerodynamic members 506 without interference between the container shell and the aerodynamic members 506. Frame 502 and/or spacer blocks 504 can be configured such that when adjacent container shells are in contact with the spacer blocks 504, the container shells have a desired alignment with respect to one another. One example of a desired alignment that can be provided by the frame 502 and/or spacer blocks 504 is aligning the adjacent container shells such that the respective major axes of the adjacent container shells are substantially parallel to one another.

Spacer blocks 504 can be attached to or provided integrally with frame 502. The spacer blocks 504 can include a spacer body 508 configured to provide an upper contact surface 510 and lower contact surface 512. Upper contact surface 510 is configured to at least partially support a first one of two adjacent container shells in a stack of container shells, for example when the container shells are stacked within a blast freezer. The upper contact surface 510 is configured to contact a bottom surface of the container shell higher up in the stack of container shells. Lower contact surface 512 is provided opposite the upper contact surface 510. Lower contact surface 512 is configured to contact a second one of the two adjacent container shells, different from the container shell contacting upper contact surface 510. Lower contact surface 512 is configured to contact a top surface of the container shell lower in the stack of container shells. In an embodiment, the respective planes of upper contact surface 510 and lower contact surface 512 are substantially parallel. Spacer body 508 can be configured such that the upper contact surface 510 and the lower contact surface 512 are separated by a predetermined distance, with the predetermined distance being based on desired flow characteristics for a flow between the adjacent container shells, such as a flow of cold air provided in a blast freezer. Examples of flow characteristics on which the predetermined distance can be based include, as non-limiting examples, pressure drop, flow rate, boundary layer thickness, or the like.

Aerodynamic member 506 can be attached to or formed integrally with frame 502 such that aerodynamic member 506 is positioned at an end of a container shell used with assembly 500. The aerodynamic member 506 can be positioned such that the aerodynamic member covers the end of the container shell from the perspective of an airflow being directed over said container shell. In an embodiment, the apparatus 500 can provide an aerodynamic member 506 at an end of the container shell facing towards a source of an airflow. In an embodiment, the apparatus 500 can provide an aerodynamic member 506 at an end of the container shell facing towards the source of the airflow. Aerodynamic member 506 includes an air control surface 514 on at least one external surface of the aerodynamic member 506. The air control surface 514 can be any suitable shape for affecting an airflow over a container shell used with apparatus 500, so as to achieve improved or desired properties of said airflow. Air control surface 514 can be configured to deflect, direct, divert, divide, combine, and/or otherwise affect the airflow over the container shell, for example when an airflow is provided over the container shell by a blast freezer, thawing apparatus, refrigerator, or the like. Air control surface 514 can be configured to, for example, reduce a boundary layer of airflow over the container shell compared to the boundary layer when the same airflow is provided over the same container shell in the absence of the aerodynamic member 506. A non-limiting example of air control surface 514 is the curved outer surface of aerodynamic member 506 as shown in FIG. 5.

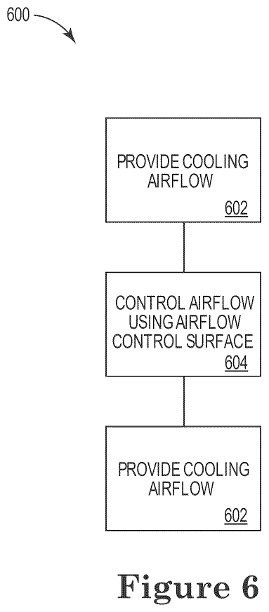

FIG. 6 shows a method of blast freezing a bioprocess material. Method 600 includes providing a cooling airflow to a container shell 602. The cooling airflow is affected by an airflow control surface at 604. Optionally, the cooling airflow can pass through a gap defined by one or more spacers between container shells at 606.

A cooling airflow is provided to a container shell at 602. The cooling airflow can be provided by any suitable source, such as a blast freezer, a refrigerator, or any other device capable of providing the cooling airflow. The device providing the cooling airflow at 602 can be configured to contain one or more of the container shells, for example within an internal space of a blast freezer or refrigerator. The cooling airflow can have any temperature suitable for cooling or freezing the contents of the container shell.

The cooling airflow is affected by an airflow control surface at 604. The airflow control surface can be provided on an aerodynamic member provided on or near the container shell. In an embodiment, the aerodynamic member is attached to or formed integrally with the container shell, such as aerodynamic member 108 described above and shown in FIG. 1. In an embodiment, the aerodynamic member is included in an assembly separate from the container shell, such as assembly 500 described above and shown in FIG. 5. The aerodynamic member can provide the airflow control surface upstream of the body of the container shell relative to the cooling airflow directed towards the container shell at 602. The airflow control surface can affect the airflow by deflecting, directing, diverting, dividing, combining, or otherwise affecting said airflow. The airflow control surface can be configured to affect the cooling airflow at 604 such that a boundary layer thickness of the cooling airflow over the container shell is relatively reduced compared to the thickness of the boundary layer that would result from the cooling airflow passing over a container shell in the absence of the airflow control surface.

The cooling airflow can pass through a gap between container shells defined by one or more spacers at 606. In an embodiment, the spacers can be attached to or formed integrally with the container shell, such as spacers 106 described above and shown in FIG. 1. In an embodiment, the spacers can be included in an assembly separate from the container shell, such as assembly 500 described above and shown in FIG. 5. The spacers can be configured to provide a gap of a predetermined size between adjacent container shells, which the cooling airflow passes through at 606. The predetermined size can be selected based on flow characteristics for the cooling airflow, such as boundary layer thickness, flow rate, pressure differentials, combinations thereof, or the like.

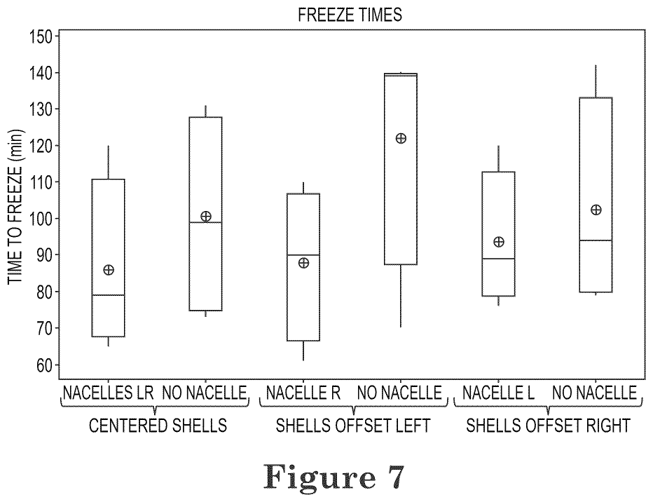

FIG. 7 shows a box plot of freezing times for bags in freezing containers according to an embodiment. The box plot is based on testing where freezing containers containing bags are placed within a convection style freezer set to −80° C. The containers are placed within the freezer, either on the left side, on the right side, or in a center. Nacelles according to embodiments are attached to the containers in the experimental trials as the aerodynamic members, whereas the containers are used without any additional aerodynamic member in the “no nacelle” control trials. In both the “nacelle” and the “no nacelle” trials, the containers are spaced apart by spacers, with the same spacers being used in each trial. The characteristes and contents of the bags are the same across all trials. The time to freeze bags within the freezing containers is measured, and the freezing time data is provided in the box plot of FIG. 7.

As shown in the box plot of FIG. 7, Regardless of position within the freezer (center, offset left, or offset right), the trials where the aerodynamic members are provided towards the source of the airflow (Nacelle LR/R/L) show lower average freezing times and also show narrower ranges for the freezing times. The reduction in average freezing time and reduction in the variance thereof show advantageous improvement of heat exchange between the air being circulated in the freezer and the contents of the containers when the aerodynamic members are provided in addition to spacers between the containers.

### Aspects

It is understood that any of aspects 1-9 can be combined with any of aspects 10-18 or 19-20. It is understood that any of aspects 10-18 can be combined with any of aspects 19-20.

Aspect 1. An apparatus, comprising:
a shell, including a front face configured to face a source of an airflow in a blast freezer and a rear face, opposite the front face; an upper surface, and a lower surface; and
an aerodynamic member positioned at one of the front face or the rear face, the aerodynamic member configured to present an airflow control surface on said one of the front face or the rear face.

Aspect 2. The apparatus according to aspect 1, further comprising one or more spacers positioned at an upper surface and/or a lower surface of the shell.

Aspect 3. The apparatus according to aspect 2, wherein the one or more spacers are configured to separate the shell from an adjacent shell by a predetermined distance.

Aspect 4. The apparatus according to any of aspects 2-3, wherein the one or more spacers are provided on the lower surface of the shell.

Aspect 5. The apparatus according to any of aspects 1-4, wherein the aerodynamic member is formed integrally with the shell.

Aspect 6. The apparatus according to any of aspects 1-4, wherein the aerodynamic member is attached to the shell.

Aspect 7. The apparatus according to any of aspects 1-6, wherein the aerodynamic member is formed of a polymer.

Aspect 8. The apparatus according to any of aspects 1-7, wherein the aerodynamic member includes a hollow cavity.

Aspect 9. The apparatus according to any of aspects 1-8, wherein the aerodynamic member is positioned at the front face.

Aspect 10. An apparatus, comprising:
a shell, the shell including a front face configured to face a source of an airflow in a blast freezer and a rear face, opposite the front face; an upper surface, and a lower surface; and
one or more spacers provided on the shell, the one or more spacers configured to separate the shell from an adjacent shell.

Aspect 11. The apparatus according to aspect 10, wherein the one or more spacers are configured to separate the shell from said adjacent shell by a distance in a range from 0.5 inches to 4 inches.

Aspect 12. The apparatus according to any of aspects 10-11, wherein the one or more spacers are provided on the lower surface of the shell.

Aspect 13. The apparatus according to any of aspects 10-12, further comprising an aerodynamic member positioned at one of the front face or the rear face, the aerodynamic member configured to present an air control surface on said one of the front face or the rear face.

Aspect 14. The apparatus according to aspect 13, wherein the aerodynamic member is formed integrally with the shell.

Aspect 15. The apparatus according to aspect 13, wherein the aerodynamic member is attached to the shell.

Aspect 16. The apparatus according to any of aspects 13-15, wherein the aerodynamic member is formed of a polymer.

Aspect 17. The apparatus according to any of aspects 13-16, wherein the aerodynamic member includes a hollow cavity.

Aspect 18. The apparatus according to any of aspects 13-17, wherein the aerodynamic member is positioned at the front face.

Aspect 19. An apparatus, comprising:
an aerodynamic member including an air control surface; and
one or more spacers configured to contact a shell, wherein the apparatus is configured to accommodate the shell such that the aerodynamic member is provided at a front face or a rear face of the shell.

Aspect 20. The apparatus according to aspect 19, wherein the one or more spacers are further configured to contact a second shell, such that the shell and the second shell are spaced apart by a predetermined distance, and the shell and the second shell are oriented in a same direction.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. An apparatus, comprising:
a shell, including a front face configured to face a source of an airflow in a blast freezer and a rear face, opposite the front face; an upper surface, and a lower surface; and
an aerodynamic member positioned at one of the front face or the rear face, the aerodynamic member configured to present an airflow control surface on said one of the front face or the rear face.

2. The apparatus of claim 1, further comprising one or more spacers positioned at an upper surface and/or a lower surface of the shell.

3. The apparatus of claim 2, wherein the one or more spacers are configured to separate the shell from an adjacent shell by a predetermined distance.

4. The apparatus of claim 2, wherein the one or more spacers are provided on the lower surface of the shell.

5. The apparatus of claim 1, wherein the aerodynamic member is formed integrally with the shell.

6. The apparatus of claim 1, wherein the aerodynamic member is attached to the shell.

7. The apparatus of claim 1, wherein the aerodynamic member is formed of a polymer.

8. The apparatus of claim 1, wherein the aerodynamic member includes a hollow cavity.

9. The apparatus of claim 1, wherein the aerodynamic member is positioned at the front face.

10. An apparatus, comprising:
a shell, the shell including a front face configured to face a source of an airflow in a blast freezer and a rear face, opposite the front face; an upper surface, and a lower surface; and
one or more spacers provided on the shell, the one or more spacers configured to separate the shell from an adjacent shell.

11. The apparatus of claim 10, wherein the one or more spacers are configured to separate the shell from said adjacent shell by a distance in a range from 0.5 inches to 4 inches.

12. The apparatus of claim 10, wherein the one or more spacers are provided on the lower surface of the shell.

13. The apparatus of claim 10, further comprising an aerodynamic member positioned at one of the front face or the rear face, the aerodynamic member configured to present an air control surface on said one of the front face or the rear face.

14. The apparatus of claim 13, wherein the aerodynamic member is formed integrally with the shell.

15. The apparatus of claim 13, wherein the aerodynamic member is attached to the shell.

16. The apparatus of claim 13, wherein the aerodynamic member is formed of a polymer.

17. The apparatus of claim 13, wherein the aerodynamic member includes a hollow cavity.

18. The apparatus of claim 13, wherein the aerodynamic member is positioned at the front face.

19. An apparatus, comprising:
an aerodynamic member including an air control surface; and
one or more spacers configured to contact a shell, wherein the apparatus is configured to accommodate the shell such that the aerodynamic member is provided at a front face or a rear face of the shell.

20. The apparatus of claim 19, wherein the one or more spacers are further configured to contact a second shell, such that the shell and the second shell are spaced apart by a predetermined distance, and the shell and the second shell are oriented in a same direction.
